(19) United States
(12) Patent Application Publication
(10) Pub. No.: US 2011/0015529 A1
(43) Pub. Date: Jan. 20, 2011

(54) METHYLENE BLUE BASED FIBRED FLUORESCENCE MICROSCOPY

(75) Inventors: Benjamin Abrat, Paris (FR); Charlotte Cave, Paris (FR); Alexandre Loiseau, Paris (FR); Jerome Lopez, Paris (FR); Anne Osdoit, Paris (FR); Bertrand viellerobe, Nogent sur Marne (FR)

Correspondence Address: YOUNG & THOMPSON, 209 Madison Street, Suite 500, Alexandria, VA, 22314, US

(73) Assignee: MAUNA KEA TECHNOLOGIES, Paris (FR)

(21) Appl. No.: 12/294,672

(22) PCT Filed: Mar. 23, 2007
(86) PCT No.: PCT/FR2007/000490

§ 371 Date: Oct. 4, 2010

(30) Foreign Application Priority Data
Mar. 31, 2006 (FR) 06/02793

(51) Int. Cl. A61B 6/00

(52) U.S. Cl. 600/478

(57) ABSTRACT

The invention relates to a method for the fabric using an acquisition system that includes at acquisition of in-vivo fluorescence imaging from a least one optical fibre exciting the fabric by scanning light beam. According to the invention, the system is used to detect fluorescence signals emitted by the Methylene Blue present in the fabric.

The present invention relates to a method for the in-vivo acquisition of fluorescence imaging of a tissue using an acquisition system comprising at least one optical fibre exciting the tissue by light beam.

A particularly useful application of the present invention is in the field of in vivo medical imaging. To this end, a fibred system is used which makes it possible, by means of data processing, to obtain an image constructed point by point in real time. Such a system makes it possible to observe and analyze a biological tissue in vivo in situ in real time, in particular accessible via the operating channel of an endoscope or integrated with the endoscope.

Generally, such a system can comprise a processing unit capable of generating an image from electrical signals originating from photodetectors. These photodetectors receive fluorescence signals originating from the tissue and carried by at least one optical fibre. The system also comprises scanning means in order that the excitation beam can carry out a point by point scan of the imaged field in the tissue.

In fluorescence imaging, a photon excites a molecule. The de-excitation of the latter causes a fluorescent photon to be emitted. The energy of the exciting photon corresponds exactly to the quantity of energy required to take the molecule to a given excited state.

In general, the observed fluorescence can come from an exogenous compound (typically an administered dye) or from an endogenous compound which is either produced by cells (transgenic type dye) of a biological tissue, or naturally present in the cells (autofluorescence). In particular, when an exogenous compound intended to be administered is used, the use of a dye having a strongly fluorescent character is generally accepted and recommended. Thus a laser having low power (excitation energy) can advantageously be used, the quantity of fluorescent photons being easily sufficient to produce high-quality images.

Generally, the following dyes are used which are known for their fluorescence properties: rhodamines, fluoresceins, cyanin, etc.

However, the problem with such dyes is that they are costly and for the most part toxic.

The object of the present invention is a simplified method for in vivo imaging, which is inexpensive and allows rapid acquisition.

At least one of the above objects is achieved with a method for the in-vivo acquisition of fluorescence imaging of a tissue using an acquisition system comprising at least one optical fibre exciting the tissue by light beam scanning. According to the invention, said system is used for the detection of fluorescence signals emitted by methylene blue present in the tissue.

The present invention is in particular noteworthy for the fact that methylene blue is used for its fluorescence properties in a fibre system for in vivo imaging. This system can in particular be of a confocal type. The confocal character is obtained using a spatial filtering making it possible to detect a return signal originating only from the excited point of the tissue and taking the same optical path as the excitation signal.

The use of methylene blue, or methylthionine chloride, goes against a widely accepted prejudice according to which the dye must have large fluorescence capacities. On the contrary, in the method according to the present invention, ease of use has been given precedence over the efficiency of the dye in terms of fluorescence. This method makes it possible to simplify the process of acquisition of fluorescence imaging by using a substance which is in common use in the medical field, but for different purposes. The pharmacological properties for which methylene blue is generally used are the following: antidote, stain, antiseptic, or also diagnostic agent.

Moreover, and more particularly, methylene blue is used in endoscopy as a “vital stain”. This is then called “chromoendoscopy” or “chromoscopy”. Methylene blue is also used as a contrast agent in a new technique called “endocytoscopy”. In this case, it is used as a dye only. Methylene blue is also used as a contrast agent for photodiagnosis. In all these cases of use as a vital stain, dye or also contrast agent, the fluorescence properties of methylene blue are not used.

Methylene blue is therefore in common use, in particular as an antiseptic. For this reason, it is therefore possible to use methylene blue on a tissue during a previous step of antiseptic treatment, and profit from its presence on the tissue to acquire a fluorescence image of the tissue. The advantages in terms of cost and time resulting from the method are immediately apparent, since it is no longer necessary to administer another fluorophore.

According to an advantageous embodiment of the invention, the acquisition system used is a confocal endomicroscopy system (microscopy during endoscopy).

According to an advantageous feature of the invention, the excitation light beam is a laser beam. Moreover, the wavelength of the excitation light beam can be comprised between 600 nm and 680 nm. The use of a confocal endomicroscopy system with a suitable wavelength makes it possible to obtain images at the microscopic level.

Advantageously, during a use on human tissues in particular, the concentration of methylene blue can be substantially equal to 0.5%. Such a value is recommended particularly to combat the toxicity of methylene blue. Nevertheless, in the present case, by best exploiting the fluorescence properties of methylene blue by laser illumination in particular, phototoxicity is produced. To combat this, it is also possible to provide for the use of a maximum methylene blue concentration equal to 0.5%. In this case, the power of the excitation laser beam and the sensitivity of the acquisition system are adapted so as to detect a fluorescence signal of a sufficiently high level to allow the acquisition system to produce an image. By way of example, the power of the excitation laser beam is less than or equal to 10 mW for one microsecond of illumination. More particularly, the power of the laser is adapted so that it does not reach the phototoxicity threshold of methylene blue, typically between 1 and 5 mW on the tissue.

Also according to the invention, it is possible to advantageously use methylene blue concentrations between 0.1% and 0.01% for a laser power comprised between 10 and 15 mW. This in particular also makes it possible to obtain good quality images.

According to an advantageous feature of the invention, the acquisition system is equipped with a fibre bundle constituted by a plurality of optical fibres which are illuminated in turn, each fibre being adapted to carrying the excitation signal and the back-emitted fluorescence signal. The system according to the invention makes it possible to produce a remote in vivo in situ fluorescence image, with a microscopic resolution. The fibre bundle has a flexibility and a size which allow an endoscopy application, in particular by insertion in an operating channel. The field to be imaged in the tissue can be scanned point by point or line by line by the excitation signal.

Preferably, the light beam scans the tissue in order to acquire at least twelve images per second in real time.

According to another feature of the invention, an application of the method as defined above is proposed for the acquisition of images of cell nodes in confocal endomicroscopy.

The present invention will be better understood and other advantages will appear in the light of the following description of an embodiment, this description referring to the attached drawings in which:

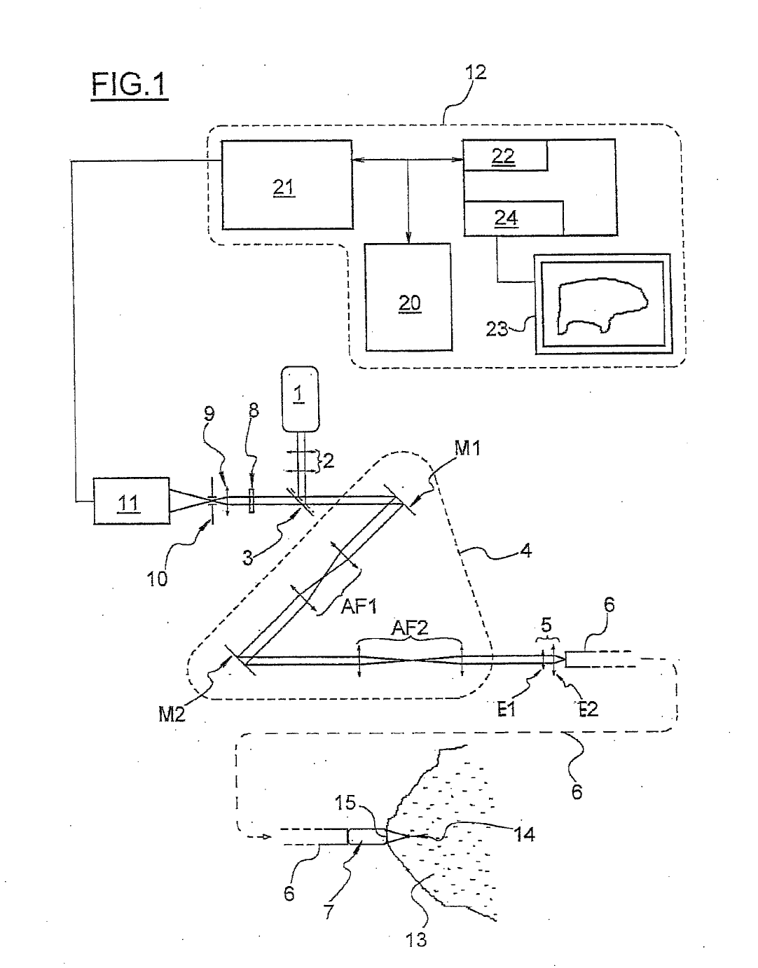

FIG. 1 diagrammatically represents said chosen embodiment; and

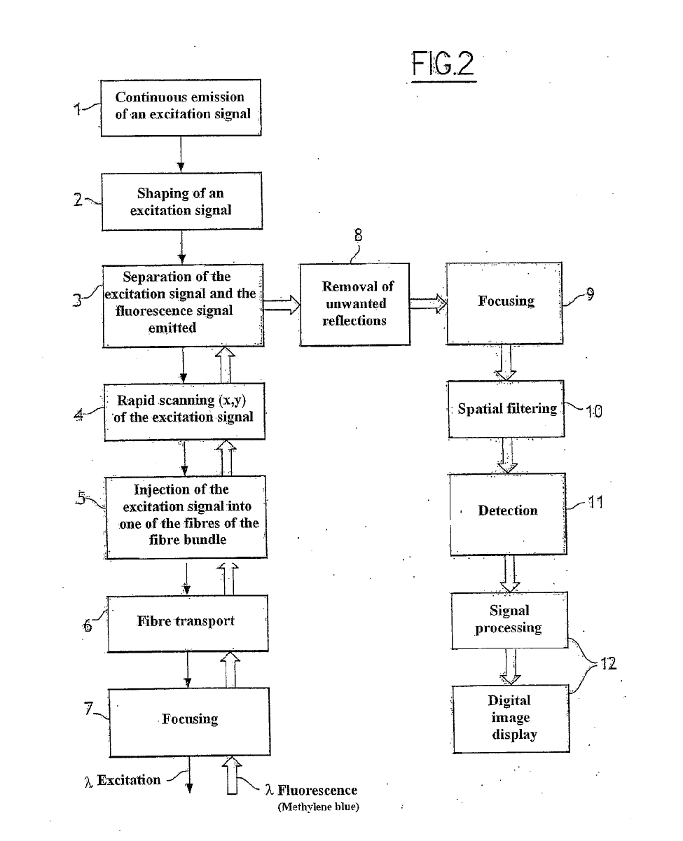

FIG. 2 is a flow chart of the apparatus according to the invention.

Although the invention is not limited thereto, a confocal microscopy fluorescence imaging system based on methylene blue will now be described. The acquisition system can consist of one of the apparatuses (with or without optical head) described in the patent WO 2004/008952. But any other suitable fluorescence apparatus can be used.

Item 13 is a biological tissue into which methylene blue has been injected in order to stain the cell nuclei. For a human tissue for example, care is taken not to exceed the concentration of 0.5%.

According to the example chosen and shown in FIGS. 1 and 2, the apparatus comprises:
a light source 1;
shaping beam excitation means 2;
wavelength separation means 3;
scanning means 4;
beam injection means 5;
an fibre bundle 6 constituted by flexible optical fibres;
a focusing optical head 7;
excitation beam rejection means 8;
means 9 of focusing the fluorescence signal;
means 10 of spatial filtering of the fluorescence signal;
means 11 of detecting the fluorescence signal; and
means 12 for electronic data processing of the detected fluorescence signal and display.

These different elements are detailed below.

The light source 1 is a laser emitting on an excitation wavelength comprised between 600 nm and 680 nm making it possible to excite methylene blue, for example 635 nm. For optimizing the injection into one of the fibres of the fibre bundle 6, the excitation beam is circular so as to be able to inject a fibre having a section which is also circular and, to optimize the injection rate, the laser is preferably longitudinal single mode in order to have the best possible wavefront for injection into a weakly multimode optical fibre. The laser emits in a continuous and stable fashion (lowest possible noise, <1%). The available output power is of the order of 20 mW. By way of example, it is possible to use a quantum well laser (VCSEL), a diode laser or also a gas laser such as helium-neon (He—Ne).

The means 2 shaping the excitation laser beam are placed at the exit from the source 1. They are constituted by an afocal optical magnification system different from 1, composed of two lenses L1 and L2 which make it possible to alter the diameter of the laser beam. The magnification is calculated such that the diameter of the beam is adapted to the injection means 5 into a fibre.

The shaped excitation laser beam is then directed towards the means 3 provided for separating the excitation and fluorescence wavelengths. This is for example a dichroic filter having a transmission efficiency of 98 to 99% of the excitation wavelength and which therefore substantially reflects the other wavelengths. The fluorescence signal, returning along the same path as the excitation signal (confocal character), will thus be sent almost entirely towards the detection path (8-11). The rejection means 8 placed on the detection path serve to totally eliminate the 1 to 2% of unwanted reflections at the excitation wavelength 635 nm which travel towards the detection path (for example a rejection filter at 635 nm or a band pass filter allowing a transmission for example only between 640 and 800 nm).

The scanning means 4 then pick up the excitation beam. According to the example chosen and shown in FIG. 1, these means comprise a mirror M1 resonating at 4 KHz serving to deflect the beam horizontally and thus produce the lines of the image, a galvanometric mirror M2 at 15 Hz serving to deflect the beam vertically and thus produce the frame of the image; and two afocal systems having unitary magnification, AF1 situated between the two mirrors and AF2 situated after the mirror M2, these afocal systems being used to conjugate the planes of rotation of the two mirrors M1 and M2 with the plane of injection in one of the fibres. According to the invention, the scanning speed is fixed to allow an observation in real time of the tissues in vivo in situ. To this end, the scanning must be sufficiently rapid for there to be at least 12 images per second displayed on the screen for a display mode of 640×640 pixels corresponding to the slowest mode. For display modes having fewer pixels, the number of images acquired per second will thus always be greater than 12 images per second. By way of a variant, the scanning means can comprise in particular a rotary mirror, integrated components of the MEMs type (X and Y scanning mirrors), or an acousto-optical system.

The excitation beam deflected on leaving the scanning means is directed towards the optical means 5 in order to be injected into one of the fibres of the fibre bundle 6. These means 5 are in this case constituted by two optical assemblies E1 and E2. The first optical assembly E1 makes it possible to partially correct the optical aberrations at the field edge of the scanning means 4, the injection thus being optimized over all of the optical field (at the centre as at the edge). The second optical assembly E2 is intended to carry out the actual injection. Its focal length and numerical aperture have been chosen to optimize the rate of injection into the optical fibres of the guide 6. According to an embodiment making it possible to obtain the criterion of achromaticity, the first assembly E1 is constituted by a doublet lens, and the second assembly E2 by two doublet lenses followed by a lens situated close to the image guide. By way of a variant, this injection optic could be constituted by any other type of standard optics, such as for example two triplets, or graded-index lenses or a microscope lens (more costly, however).

The fibre bundle 6 is constituted by a very large number of flexible optical fibres (a few tens of thousands), for example 30,000 fibres of 2 μm diameter and spaced apart by 3.3 μm. In practice, it is possible to use either all of the fibres of the image guide, or a chosen subassembly of these fibres, for example centred.

On leaving the optical fibre, the excitation laser beam is focused by the optical head 7 in the specimen 13 at a point 14 situated at a given depth situated between a few tens of μm and approximately a hundred μm, in relation to the surface 15 of the specimen in contact with which it is intended for the optical head to be placed. This depth can be for example 40 μm. The optical head thus makes it possible to focus the flux leaving the fibre bundle in the specimen, but also to collect the fluorescence flux returning from the specimen. The optical head has a magnification of 2.4 and a numerical aperture on the specimen of 0.5. These two parameters are chosen so that the return signal materializes only in the optical fibre which has transmitted the excitation signal and not in the adjacent fibres and thus preserves the confocal filtering using one fibre. With these magnification and numerical aperture values, the axial resolution is of the order of 10 μm and the lateral resolution is of the order of 1.4 μm. The numerical aperture is also chosen so as to optimize the number of photons recovered which must be the maximum possible. The optical head can be constituted by standard optics (doublet, triplet, aspherical) and/or graded-index lenses (GRIN) having an optical quality and chromatism adapted to the confocality, i.e. minimizing the optical aberrations, which otherwise would lead in particular to degradations on the field depth and as a result on the axial resolution of the apparatus. In operation, the optical head is intended to be placed in contact with the specimen 13. The latter is a biological tissue or a cell culture. The expression of the fluorescence is realized by the methylene blue, which re-emits photons over a spectral band from several tens of nanometres to more than one hundred or so nanometres. On the detection path, the fluorescence signal, on leaving the rejection filter 8, is then focused by the means 9, constituted for example by a detection lens, into a filtering hole of the spatial filtering means 10. The focal length of the detection lens is calculated so that the fluorescence signal from a fibre is of the same size or slightly less than that of the filtering hole. The latter makes it possible to preserve only the fluorescence light from the fibre illuminated by the incident beam. It makes it possible to reject the light which could have been coupled in the fibres adjacent to the one which is illuminated. The size of the hole is calculated so that the image of a fibre fits perfectly therein. Here, it is 20 μm. Moreover, in order to optimize the quantity of photons passing through the filtering hole, and therefore the flux detected, the scanning means 4, the injection means 5, the means of focusing 7 of the optical head, and the detection means 8, 9 and 10 are adapted to the detected fluorophore: these means are chosen to be sufficiently achromatic to collect photons over the widest emission band of the fluorophore which is methylene blue.

The detection means 11 have a maximum sensitivity to the fluorescence wavelengths of methylene blue. For example an avalanche photodiode (APD) or a photo-multiplier can be used. Generally, all of the chain of acquisition is optimized (optical head, waveguide, detector, image processing and electronics software) so as to detect a few hundred pico watts of fluorophore over an interval of approximately 80 nanoseconds. Thus only a few photons of methylene blue are detected with a system operating in real time.

Moreover, in order to obtain an image in real time, the pass band is preferably chosen to optimize the integration time of the fluorescence signal. It is 2 MHz, which corresponds to the minimum sampling frequency of the fibre bundle with an integration time optimized on each pixel.

The electronic data processing means 12, for control, analysis and digital processing of the detected signal and for display, include the following cards:
a synchronisation card 20 the functions of which are:
to control the scanning in a synchronized manner, i.e. the movement of the line M1 and frame M2 mirrors;
to know at all times the position of the laser spot scanned in this way; and
to manage all the other cards via a microcontroller which can itself be controlled;
a detector card 21 which comprises an analogue circuit which carries out in particular an impedance adaptation, an analogue-to-digital converter, then a programmable logic component (for example an FPGA circuit) which shapes the signal;
a digital acquisition card 22 which makes it possible to process a flow of digital data at a variable frequency and display it on a screen 23;
a graphics card 24.

By way of a variant, a single card can be used, which combines the functionalities of these different cards.

The image processing takes place as follows.

When an fibre bundle is placed in the apparatus, a first operation is carried out to recognize the pattern of the fibres in the image guide, and therefore to know the actual location of each fibre intended to be used.

The following operations are also carried out prior to the use of the apparatus:
determination of the injection rate adapted to each fibre, using a homogenous specimen, this injection rate being able of vary from one fibre to another; and
measurement of the background image, carried out without a specimen.

These two operations can be carried out regularly according to the frequency of use of the apparatus. The results obtained will be used to adjust the digital signal on leaving the detector card while in operation.

In operation, according to the invention, 2 groups of processing are carried out on the digital signal on leaving the detector card:

The first group consists firstly of adjusting the digital signal in particular to take account of the actual rate of injection of the fibre from which said signal originated and to subtract from it the part of the flux corresponding to the background image. This makes it possible to process only a signal actually corresponding to the specimen observed. For this processing group, a standard calculation algorithm is used which can be optimized to comply with real-time constraints if appropriate.

The second group then consists of reconstructing, from the adjusted signal, the digital image which will be displayed by the practitioner. The aim of the processing carried out is to provide a reconstituted digital image for display which is not simply a mosaic of image elements each corresponding to an adjusted digital signal of a fibre placed side by side, but to provide a reconstituted digital image which no longer shows the fibres. To this end, an algorithm is used which is intended to carry out a certain number of operations on each pixel, the algorithm being chosen to comply with the real-time constraints, i.e. it must represent a good compromise between the complexity of the operations required, the quality of the result that can be obtained and the calculation time. By way of example, a Gaussian low-pass filtering algorithm can be used.

The apparatus operates as follows. The source 1 produces a circular parallel excitation beam at λ=635 nm, which is then shaped in the afocal system 2 in order to give it the appropriate size for the best possible injection into the core of a fibre. This beam is then sent to the dichroic separation system 3 which reflects the excitation wavelength. The incident beam is then deflected angularly in time in the two spatial directions by the optometric scanning mirror system 4, and injected using the optical injection means 5 into one of the fibres of the fibre bundle 6. The electronic means 12 serve to control the injection at a given moment of one of the optical fibres of the fibre bundle by angularly deflecting the beam using the mirrors, point by point for a given line, and line after line, to constitute the image. On leaving the guide, the light emerging from the injected fibre is focused in the specimen using the optical head 7 at a point situated at a given depth situated approximately between a few tens of μm and approximately one hundred μm. Using the scanning, the specimen is illuminated point by point. At each moment, the spot illuminating the tissue then emits a fluorescence signal which has the feature of being shifted towards longer wavelengths. This fluorescence signal is captured by the optical head 7, then follows the reverse path of the excitation beam as far as the dichroic filter 3 which will transmit the fluorescence signal to the detection path. The unwanted reflections occurring at the excitation wavelength will then be rejected by the rejection filter 8. Finally, the fluorescence signal is focused into the filtering hole 10 in order to select only the light from the excited fibre and the photons are detected by the avalanche photodiode 11. The detected signal is then digitized and corrected. The detected signals are processed sequentially in real time, using the image processing described above, to allow the real-time construction of an image and display on the screen.

The method according to the present invention can be used in particular for the observation of human tissue, limiting the concentration of methylene blue in the tissue: a low concentration of the order of 0.5%; by limiting the power of the laser beam: a low power of the order of 10 mW/μs; and increasing the sensitivity of detection of the system so as to detect a maximum of fluorophore.

Of course, the invention is not limited to the examples which have just been described, and numerous adjustments can be made to these examples without exceeding the scope of the invention. In particular, it is possible to use a system comprising a single optical fibre or a “bundle” of optical fibres, a point to point or line to line scanning, a distal or proximal scan, with or without optical head, for fluorescence or reflectance.

1. Method for the in vivo acquisition of fluorescence imaging of a tissue using an acquisition system comprising at least one optical fibre exciting the tissue by scanning of a light beam, characterized in that said system is used for the detection of fluorescence signals emitted by methylene blue present in the tissue.

2. Method according to claim 1, characterized in that said system is a confocal endomicroscopy system (microscopy during endoscopy).

3. Method according to claim 1, characterized in that the excitation light beam is a laser beam.

4. Method according to claim 1, characterized in that the wavelength of the excitation light beam is comprised between 600 nm and 680 nm.

5. Method according to claim 1, characterized in that the concentration of methylene blue is approximately equal to 0.5%.

6. Method according to claim 1, characterized in that a maximum concentration of methylene blue equal to 0.5% is used, and in that the power of the excitation laser beam and the sensitivity of said system are adapted to detect a fluorescence signal at a sufficiently high level to allow the system to produce an image.

7. Method according to claim 1, characterized in that the power of the excitation laser beam is less than or equal to 10 mW for a microsecond of illumination.

8. Method according to claim 6, characterized in that a concentration of methylene blue comprised between 0.1 and 0.01% is used, and in that the light beam is a laser beam having a power comprised between 10 and 15 mW.

9. Method according to claim 1, characterized in that said system is equipped with a fibre bundle constituted by a plurality of optical fibres which are illuminated in turn, each fibre being adapted to carrying the excitation signal and the back-emitted fluorescence signal.

10. Method according to claim 1, characterized in that the light beam scans the tissue so as to acquire at least twelve images per second in real time.

11. Application of the method according to claim 1 for the acquisition of images of cell nuclei in confocal endomicroscopy.

12. Method according to claim 2, characterized in that the excitation light beam is a laser beam.
